(19) United States
(12) Patent Application Publication
(10) Pub. No.: US 2011/0305648 A1
(43) Pub. Date: Dec. 15, 2011

(54) Methods of Preparing Personal Care Compositions

(76) Inventors: Dawn Renee KNAPEK, West Chester, OH (US); Lowen Robert Morrison, JR., Cincinnati, OH (US); Marco Caggioni, Cincinnati, OH (US); William Joseph Worley, Mason, OH (US); Douglas Allan Royce, Sunman, IN (US); Jennifer Elaine Hilvert, Cincinnati, OH (US)

(21) Appl. No.: 13/156,862

(22) Filed: Jun. 9, 2011

Related U.S. Application Data
(60) Provisional application No. 61/353,034, filed on Jun. 9, 2010.

(51) Int. Cl. A61K 8/46; A61K 8/81; C11D 3/60; A61Q 5/00; A61Q 11/00; A61K 8/84; A61Q 17/04

(52) U.S. Cl. 424/56; 514/772; 424/70.6; 514/772.3; 514/772.1; 514/772.4; 424/59; 510/119; 510/137; 510/158; 510/276; 510/108

(57) ABSTRACT

Disclosed herein are embodiments of a method of predicting and adjusting the viscosity of consumer product compositions prior to their final formulation. The prediction aides the determination of the amount of a viscosity modifier to introduce in the manufacture of the final formulation to ensure the compositions have viscosities that satisfies consumer preferences, among other things.

### CROSS REFERENCE TO RELATED APPLICATION

This application claims the benefit of U.S. Provisional Application No. 61/353,034 filed Jun. 9, 2010.

### FIELD OF THE INVENTION

The disclosure generally relates to methods employed in the manufacture of personal care compositions and, more specifically, to methods employed to provide personal care compositions with desirable viscosities.

### BACKGROUND OF THE INVENTION

Personal care compositions generally include shampoos, shower gels, liquid hand cleansers, liquid dental compositions, skin lotions and creams, hair colorants, facial cleansers, and fluids intended for impregnation into or on wiping articles (e.g., baby wipes). These compositions are often manufactured to meet consumer preferences, such as consistency, feel, and other aesthetic qualities. Further, these compositions are often manufactured to endure extended periods of storage and transportation without any meaningful deterioration in performance or aesthetic qualities.

These compositions are commonly manufactured in batch processes where surfactants, co-surfactants, and various other ingredients are combined to form a product. At this stage, the product is typically ready for packaging (e.g., bottling in the case of shampoo) and shipment.

The product, however, may not have a viscosity suitable for consumer preferences or other downstream processing by further unit operations. Consequently, the viscosity of the product may require adjustment before further processing or packaging. In a batch process, this can result in delay, and loss of production capacity due to longer batch cycle time. In existing continuous processes for manufacturing personal care compositions, the ability to adjust viscosity once the ingredients have been mixed and a product formed is limited and results in high scrap rates if the product is expected to continue directly to a packaging stage. Further, in a single manufacturing plant, many different compositions may be made in short periods of time. These products may vary drastically from one to another. In existing continuous processes some waste is traditionally generated in changing-over product lines. Even in batch processes, the change-over introduces challenges in the manufacture of such products. In continuous processes, however, it would be desirable to reduce the amount of waste product generated between different product manufactures. Furthermore, while it would be desirable to more efficiently adjust the viscosity of the finished product in a batch process, it is particularly desirable in continuous and semi-continuous manufacture of personal care compositions to accurately predict the viscosity so that viscosity adjustment of the finished product is not necessary.

### SUMMARY OF THE INVENTION

Disclosed herein are embodiments of a method of predicting and adjusting the viscosity of consumer product compositions prior to their final formulation. According to one embodiment, the method includes combining ionic surfactant-containing feeds in a mixer to form a composition selected from the group consisting of a personal care composition, a laundry detergent composition, and a cleaning composition. The method further includes determining the concentration of ions necessary in the composition to achieve a composition viscosity. This determination step can occur either before or after the aforementioned combining step so long as it is known which ionic surfactants will be present in the composition and in what concentrations each will be present. The method further includes measuring the conductivity of one or more ionic surfactant-containing feeds upstream of the mixer, and correlating the measured conductivity to the concentration of ions present in the measured ionic surfactant-containing feeds. Thereafter, the method includes introducing a viscosity modifier to the mixer in a quantity per unit flow of the composition sufficient to achieve the composition viscosity.

According to another embodiment, a continuous method of making a personal care composition includes simultaneously combining continuous flows of ionic surfactant-containing feeds and a viscosity modifier selected from the group consisting of an ion source, a hydrotrope, and a polymeric thickener, to form a personal care composition having a viscosity in a range of about 2.5 Pascal seconds (Pa·s) to about 100 Pa·s at 25° C. and at a shear rate of 2 per second (s⁻¹). The method also includes measuring the conductivity of one or more ionic surfactant-containing feeds prior to formation of the composition, and correlating the measured conductivity to an intermediate salt concentration of each feed measured. In this method, the flow of the viscosity modifier is at a rate per unit flow of the ionic surfactant-containing feeds sufficient to achieve a salt concentration in the formed personal care composition corresponding to the viscosity range.

The personal care composition can be one selected from the group consisting of a shampoo composition, shower gel, liquid hand cleanser, liquid dental composition, skin lotion and cream, hair colorant, facial cleanser, and fluids intended for impregnation into or on wiping articles. The same embodiments, however, can be employed in the continuous manufacture of laundry detergent compositions and cleaning compositions.

Still further embodiments include methods of predicting and adjusting final composition viscosity based on conductivities of ingredient feeds. In a continuous manufacture of a composition selected from the group consisting of a personal care composition, a laundry detergent composition, and a cleaning composition, for example, the method can include measuring the conductivity of one or more ingredients employed in the manufacture, correlating the measured conductivity to an intermediate salt concentration of the composition, and mixing with the ingredients a quantity of viscosity modifier sufficient to change the intermediate salt concentration of the composition to a target salt concentration, preferably sufficient to achieve a target composition viscosity of about 2.5 Pa·s to about 100 Pa·s at 25° C. and at a shear rate of 2 s⁻¹. In this embodiment, correlating the measured conductivity to an intermediate salt concentration of the composition further includes determining the difference between the target salt concentration and the intermediate salt concentration. Further, the quantity of viscosity modifier mixed with the ingredients can be a function of this difference.

Additional features of the invention may become apparent to those skilled in the art from a review of the following detailed description, taken in conjunction with the drawing figures, the example, and the appended claims.

### BRIEF DESCRIPTION OF THE DRAWINGS

For a more complete understanding of the disclosure, reference should be made to the following detailed description and accompanying figures wherein:

FIG. 1 is process flow diagram for a continuous manufacture of a personal care composition that may employ embodiments of the inventive methods disclosed herein; and,

FIG. 2 is a graphical depiction of the profiler from a statistical analysis performed to determine a target sodium ion concentration and correlate the same to a suitable viscosity in accordance with embodiments of the inventive methods disclosed herein.

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

### DETAILED DESCRIPTION OF THE INVENTION

It has now been discovered that the conductivity of materials employed in the manufacture of a personal care composition, for example, can be used to predict the viscosity of the finished composition in the absence of any viscosity modifier. The prediction aides the determination of the amount of modifier to introduce to ensure the composition has a viscosity that satisfies consumer preferences, among other things. For a number of reasons, this is advantageous over the conventional manufacture, where a batch of the composition must first be made and thereafter modified with the introduction of a viscosity modifier before the composition is packaged. This is advantageous because the method now provides an ability to make a product in a continuous process previously impractical due to the difficulty in adjusting the viscosity only after the composition is made and immediately before the material is bottled. This prediction method is also advantageous because it minimizes waste product that would accompany changes in products made employing the conventional process equipment. This is further advantageous because it is allows the manufacture of smaller lots of personal care compositions with minimal waste product, currently smaller lots cannot be made due to the waste material generated during product line change-over.

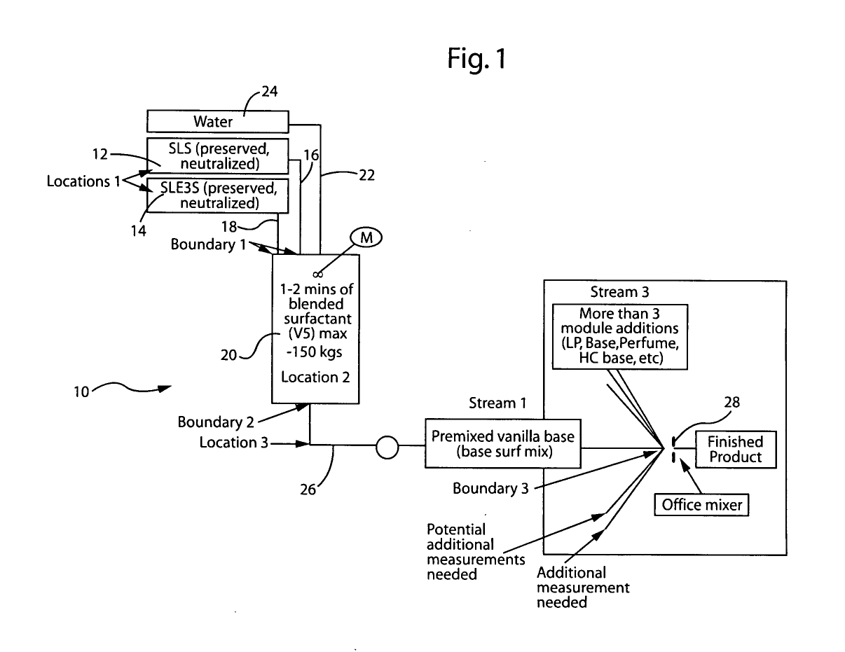

With reference to the drawing figures, wherein like reference numbers refer to the same or similar elements in the various figures, a general process flow of a process for the manufacture of a personal care composition is shown. Specifically, FIG. 1 is a diagram of the flow for a process 10 of manufacturing a shampoo composition. Generally, the process 10 includes one or more ionic surfactants supplied from tanks (or plant supply systems) 12 and 14, through supply lines 16 and 18, respectively, to a mixing vessel 20 where the surfactants are combined with water to form a base material for further use in the manufacture of the personal care composition. Water is supplied to the mixing vessel 20 through a supply line 22 and from a tank 24. Base material may exit the mixing vessel 20 through a discharge line 26 and continue in the process 10 to a mixer 28 immediately before which it is combined with additional ingredients that make up the personal care composition. The mixer 28 may be a stirred tank, in the case of a batch process, or an in-line mixing device, in the case of a continuous process, employed to collect each of the ingredients that make up the personal care composition and then combine the same to form the composition. Although the process shown in FIG. 1 is described herein as exemplary of the manufacture of a shampoo composition, the shown process can be readily employed with modifications, as necessary, in the manufacture of other personal care compositions as well as other consumer products, such as, for example, laundry detergent compositions and cleaning compositions.

A number of ingredients may be combined with the base to form the personal care composition having a viscosity consistent with the viscosity contributions made by each incoming ingredient. For a variety of reasons, that viscosity, however, may not be suitable for a finished product bottled for consumer use. Accordingly, the viscosity must be adjusted post formulation to better accommodate consumer preferences, for example. This can be accomplished by measuring the viscosity of the formed personal care composition and then directly mixing a viscosity modifier into the composition prior to bottling. As explained in more detail below, there are a number of limitations to, and disadvantages in, modifying the viscosity in this manner.

Alternatively, viscosity modifications can be accomplished with fewer limitations, and advantageously, by predicting the viscosity of the anticipated formulation and thereafter pre-adjusting the viscosity by the addition of a viscosity modifier into the formulation as it is formed to ensure a personal care composition having a viscosity better suited for consumer use. Disclosed herein are embodiments of this method.

According to one embodiment, the method includes combining ionic surfactant-containing feeds in a mixer to form a personal care composition. The method further includes determining the concentration of ions necessary in the composition to achieve a composition viscosity, preferably of about 2.5 Pascal seconds (Pa·s) to about 100 Pa·s at 25° C. and at a shear rate of 2 per second (s⁻¹). This determination step can occur either before or after the aforementioned combining step so long as it is known which ionic surfactants will be present in the personal care composition and in what concentrations each will be present. The method further includes measuring the conductivity of one or more ionic surfactant-containing feeds upstream of the mixer, and correlating the measured conductivity to the concentration of ions present in the measured ionic surfactant-containing feeds. Thereafter, the method includes introducing a viscosity modifier to the mixer in a quantity per unit flow of the composition sufficient to achieve the composition viscosity. This method can be employed to prepare laundry detergent compositions as well as cleaning compositions. Surfactants and viscosity modifiers suitable for use in accordance with this method are disclosed below.

In further embodiments of this method, the mixer can combine not only the ionic surfactant-containing feeds but also the viscosity modifier and other ingredients to form the personal care compositions. The mixer preferably is selected from the group consisting of an agitated mixer, an orifice, a homogenizer, an in-line dynamic mixer, a high pressure sonic mixer, and a static mixer. An example of an agitated mixer is one commercially available from Lotus Mixers, Inc. (Nokomis, Fla.) under the product name FJ, FJA series mixers. An example of a homogenizer is a liquid whistle, commercially available from Sonic Corp. (Stratford, Conn.) under the product name Sonolator. An example of an in-line dynamic mixer is one commercially available from Hayward Gordon (Toronto, Canada) under the product name Dynamic in line series. An example of a static mixer is one commercially available from Lotus Mixers, Inc. (Nokomis, Fla.) under the product name SL static mixer.

Continuous methods of making personal care compositions likely will employ mixers, among those listed above, for example, that are suitable for continuous processes. Thus, in a preferred embodiment, the step of combining includes continuously flowing the feeds to the mixer. These flowing feeds can optionally be introduced into the mixer simultaneously. According to another embodiment, a continuous method of making a personal care composition includes simultaneously combining continuous flows of ionic surfactant-containing feeds and a viscosity modifier selected from the group consisting of an ion source, a hydrotrope, and a polymeric thickener, to form a personal care composition having a viscosity in a range of about 2.5 Pa·s to about 100 Pa·s at 25° C. and at a shear rate of 2 s⁻¹. The method also includes measuring the conductivity of one or more ionic surfactant-containing feeds prior to formation of the composition, and correlating the measured conductivity to an intermediate salt concentration. In this method, the flow of the viscosity modifier is at a rate per unit flow of the ionic surfactant-containing feeds sufficient to achieve a salt concentration in the formed personal care composition corresponding to the viscosity range.

Still further embodiments include methods of predicting and adjusting final composition viscosity based on conductivities of ingredient feeds. In a continuous manufacture of a personal care composition, for example, the method can include measuring the conductivity of one or more ingredients employed in the manufacture, correlating the measured conductivity to an intermediate salt concentration of the composition, and mixing with the ingredients a quantity of viscosity modifier sufficient to change the intermediate salt concentration of the composition to a target salt concentration sufficient to achieve a target composition viscosity, preferably of about 2.5 Pa·s to about 100 Pa·s at 25° C. and at a shear rate of 2 s⁻¹. In this embodiment, correlating the measured conductivity to an intermediate salt concentration of the composition further includes determining the difference between the target salt concentration and the intermediate salt concentration. Further, the quantity of viscosity modifier mixed with the ingredients may be a function of this difference. The personal care composition can be one selected from the group consisting of a shampoo composition, shower gel, liquid hand cleanser, liquid dental composition, skin lotion and cream, hair colorant, facial cleanser, and fluids intended for impregnation into or on wiping articles. The same method can be employed in the continuous manufacture of laundry detergent compositions and cleaning compositions.

As stated above, the method includes determining the concentration of ions necessary in the composition to achieve a composition viscosity of about 2.5 Pascal seconds (Pa·s) to about 100 Pa·s, preferably at 25° C. and at a shear rate of 2 per second (s⁻¹). This viscosity may vary depending upon the type of composition desired. In certain embodiments of shampoo compositions, the viscosity is about 2.5 Pa·s to about 25 Pa·s under the same measuring conditions, whereas in other embodiments of shampoo compositions the viscosity is about 5 Pa·s to about 12 Pa·s under the same measuring conditions.

Generally, the viscosity of an ionic composition, such as a personal care product, is a function of the concentration of free ions (e.g., cations) present in the composition. This concentration can be determined by analysis of each of the ingredients that will be used to form the product. These ingredients are typically stored in separate vessels (or plant supply systems) and delivered as feeds into a mixer, such as those described above, that will be used to formulate the final composition. Within the storage vessel (or plant supply systems), each ingredient may be maintained or preserved in solution or dry form so that each is readily available for combination with one another to form the composition. Feeds containing ionic surfactants are expected to contain the greatest concentration of free cations. Further, those feeds containing greater amounts of ionic surfactants (or other ionic components) are expected to strongly influence the viscosity of the formed product. Consequently, careful control of these feeds can aid in the manufacture of personal care products suitable for consumer use.

Generally, when an ionic compound, such as an ionic surfactant, is dissolved in water, ions break away from the solid and spread evenly throughout the solution. Ions dissolved in a solution, such as sodium ions (Na⁺) and chlorine ions (Cr) can conduct electricity because each carries an electric charge. The conductivity of a solution, or how easily electricity passes through the solution, depends on the concentration of ions. If a large amount of sodium chloride is added to water, the solution will, of course, have large amounts of sodium ions and chlorine ions when the salt dissolves. This solution will conduct electricity well. Similarly, if small amounts of sodium chloride are added to the same volume of water, the solution will have fewer ions and, therefore, will be less conductive. Conductivity is measured in SI units of microSiemens per centimeter (μS/cm) or milliSiemens per centimeter (mS/cm). Generally, the greater the conductivity reading is, the more ions there must be in the solution.

In the context of shampoo compositions, it has been found that surfactants such as sodium lauryl sulfate (SLS) and sodium laureth sulfate (with 3 moles of ethoxylation, SL3ES) begin in a spherical micelle configuration, with little interaction between the micelles and, therefore, relatively low viscosity (10 centipoise at a shear rate of 2 per second). As the salt concentration is increased in the shampoo, from sodium chloride addition or the carry over salt from the manufacture of cocoamidopropyl betaine, the spherical micelles change into worm-like micelles. As worm-like micelles grow, they begin to entangle, and the viscosity of the shampoo increases.

As solution conductivity is a function of the concentration of ions present in the solution, and as viscosity is also a function of the concentration of ions present in the solution, viscosity and solution conductivity are functionally related to one another. This latter relationship can be advantageously exploited in the manufacture of personal care compositions by, for example, fine-tuning the viscosity of the same to more efficiently provide personal care products suitable for consumer use. For example, conductivity measurements of personal care composition ingredients responsible for major contributions of ions in the finished composition are meaningfully predictive of the viscosity of the finished composition. When those measurements are made upstream of the unit operation(s) responsible for combining the ingredients, the manufacturer can more efficiently supplement the final composition with a viscosity modifier (e.g., an electrolyte, a hydrotrope, and/or a polymeric thickener). This can be further advantageous in continuous manufacturing processes where changes in-line from one product to another require prompt adjustments in viscosity modifiers to ensure rheologically-acceptable products for consumer use without substantial waste generated during the change.

Personal care products typically will have a target viscosity range that is based on consumer preferences and the ingredients comprising the products. Although the ingredients themselves determine the viscosity of the products, the viscosity can be adjusted by minor changes in the product's composition to satisfy consumer preferences. Heretofore, those minor changes were difficult to determine and nearly impossible to undertake in continuous processes without the high risk of great waste. It has now been discovered, however, that viscosity may be conveniently predicted and quickly modified by the efficient introduction of viscosity modifiers in the manufacture. This is further described with reference to the manufacture of a shampoo composition, but it should be understood that the process can be employed in the manufacture of any personal care composition as well as in the manufacture of other consumer products such as cleaning compositions and laundry detergent compositions.

With known ingredients and concentrations thereof in the manufacture of a shampoo composition, the concentration of free cations expected in the finished product in the absence of a viscosity modifier are determined. This generally entails consideration of the lot-to-lot variations in the manufacture of surfactants and co-surfactants expected to contribute ions (e.g., cations) to the finished product. That consideration should, therefore, contemplate, the contributions of free ions from various components of the surfactants and co-surfactants. For example, a commercially available lot of sodium lauryl sulfate (SLS) may contain 0.08 wt. % to 0.5 wt. % sodium bisulfate (Na₂SO₄), 0.1 wt. % to 0.2 wt. % sodium chloride, and 0.37 wt. % to 0.6 wt. % unreacted fatty alcohol. Similarly, a commercially available lot of sodium laureth sulfate (3 moles of ethoxylation), SL3ES, may contain 0.1 wt. % to 0.6 wt. % sodium bisulfate (Na₂SO₄), 0.005 wt. % to 0.4 wt. % sodium chloride, and 0.85 wt. % to 1.1 wt. % unreacted fatty alcohol. Cocoamidopropyl betaine, a common co-surfactant used in shampoo manufacture, may contain 5.43 wt. % to 6 wt. % sodium chloride. These variances can significantly affect the quantity of cations present in the final composition and, therefore, should be taken into account. The conductivity of the base surfactants and co-surfactant composition can be used to predict the number of moles of cations present from the salts contributed by the base composition, and thereafter to determine the amount of viscosity modifier necessary to achieve a target viscosity.

The procedure generally involves determining the conductivity of the personal care composition in the absence of a viscosity modifier, a determination that also accommodates the aforementioned variances in lot-to-lot manufacture of the ion- (cation-) contributing ingredient. Once that determination has been made, a functional relationship may be recognized between the concentration of cations in the finished product to the concentration of salt and fatty alcohol in the feeds. For example, four separate experiments can be set up based on permutations of whether salt content is high or low and whether the fatty alcohol content is high or low. The concentration of free ions can be calculated in each experiment. The viscosity and conductivity of the composition in each experiment can be determined through standard viscosity and conductivity measurement and statistical analytical techniques. This information can be plotted to determine a relationship between the conductivity and viscosity of the four compositions. From this relationship, a target conductivity can be associated with a target viscosity. With this information and with the known concentration of salt ions contributed by each component, a target salt ion concentration may be determined.

Once this information has been obtained, viscosity modifiers may be included in the manufacture of shampoo compositions containing the same base surfactants and co-surfactants to ensure a viscosity within an acceptable range consistent with consumer preferences. For example, the conductivity of the major ionic surfactant-containing feeds in the manufacture can be measured, and the concentration of the free cations in those feeds can be determined. Thereafter, knowing the target conductivity necessary to achieve a suitable viscosity, and knowing the number of free cations that will illicit the target conductivity in the composition, the manufacturer can supplement the composition with a viscosity modifier (e.g., an electrolyte, a hydrotrope) to adjust the conductivity and, therefore, the viscosity of the composition being made.

The conductivity measurements may be made at various points upstream of where the ingredients are ultimately combined to form the personal care composition. A number of such points are illustrated in FIG. 1. Therein, “Location 1” is defined as a point where the anionic surfactants may be measured prior to combination and/or dilution (mixing vessel 20). Measurements preferably are made at this location for each anionic surfactant employed. The precise site for “Location 1” may be in the surfactant making system (for example in the tank for chlorosulfonic acid process or continuous line for falling film reactor), in a secondary surfactant neutralization system, in a surfactant storage device (tank or portable), in a delivery pipe (supply lines 16 and 18), or anywhere prior to delivery into the combination/dilution device (mixing vessel 20). This will desirably permit for manual or automated calculation of salt content feed necessary for each run.

“Location 2” shown in FIG. 1 is defined as a point at which the anionic surfactants are combined and/or diluted (mixing vessel 20). At this site, measurement would permit “feed forward” of a salt stream feed needed, which could change during the run if the conductivity changes. “Location 3” is defined as a point following the combination and/or dilution of multiple anionic surfactants (downstream of the mixing vessel 20), but prior to combination with other ingredients that will make up the final composition. At this site, measurement would permit “feed forward” of a salt stream feed needed, which could change during the run if the conductivity changes. Measurements at each of locations 2 and 3 would be desirable.

“Additional measurement” may be needed for all amphoteric or Zwitterionic co-surfactant feeds that contribute significant ionic components into the final composition. The site for such “Additional measurement” may be anywhere between the synthesis of the component prior to its combination with other ingredients that will make up the final composition. This “Additional measurement” is necessary regardless of the location choice for measuring the conductivity of the anionic surfactant feeds.

“Potential additional measurement” may be needed for all non-co-surfactant feeds that contribute significant ionic components (including anionic surfactant) into the final composition. Examples of such feeds include stabilizers, cocamide monoethanolamine, and high charge density cationic polymers. The site for such “Potential additional measurement” may be anywhere between the synthesis of the component prior to its combination with other ingredients that will make up the final composition. This “Potential additional measurement” is necessary regardless of the location choice for measuring the conductivity of the anionic surfactant feeds, especially where the component is expected to contribute as much as 2% active anionic surfactant to the finished composition.

The conductivity measurement is dependent upon the temperature of the measured solution. As temperature increases, the mobility of ions in the solution increases and the conductivity increases. The measurements should account for the temperature and, preferably, the conductivity of the materials should be made at consistent temperatures throughout the process. Whatever the relationship may be between conductivity and temperature, the relationship should be programmed into the transmitter or process control systems responsible for making and reporting the measurements.

Common ingredients in the manufacture of personal care compositions are disclosed below. A number of ingredients and even entrained air can influence conductivity measurements. For example, if surfactants are neutralized with citric acid, sodium citrate may be used in the case of pH overshoot. This will likely have an impact on the amount of sodium ions and conductivity of the base surfactant mix.

The term “polymer”, as used herein, shall include materials whether made by polymerization of one type of monomer or made by two (i.e., copolymers) or more types of monomers.

The term “charge density” as used herein, means the ratio of the number of positive charges on a monomeric unit of which a polymer is comprised to the molecular weight of said monomeric unit. The charge density multiplied by the polymer molecular weight determines the number of positively charged sites on a given polymer chain.

The term “alkyl” refers to a saturated or unsaturated straight or branched chain hydrocarbon group of carbon atoms, including, but not limited to, methyl, ethyl, n-propyl, isopropyl, n-butyl, isobutyl, tert-butyl, n-hexyl, and the like. C₁₋₈alkyl refers to substituted or unsubstituted alkyl groups that can have, for example from 1 to 8 carbon atoms. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halogen, aryl, heteroaryl, cycloalkyl, heterocycloalkyl, and amino. “Heteroalkyl” is defined similarly as alkyl, except the heteroalkyl contains at least one heteroatom independently selected from the group consisting of oxygen, nitrogen, and sulfur.

The term “alkylene” refers to a straight or branched alkyl group chain having two points of attachment to the rest of the molecule.

The term “alkenyl” refers to a straight or branched chain hydrocarbon group of at least two carbon atoms containing at least one carbon double bond including, but not limited to, 1-propenyl, 2-propenyl, 2-methyl-1-propenyl, 1-butenyl, 2-butenyl, and the like.

The term “alkoxy” refers to a straight or branched chain alkyl group covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “oxyalkylene” refers to an alkoxy group having two points of attachment to the rest of the molecule, one of the points being through the oxygen atom.

The term “alkoxyalkyl” refers to one or more alkoxy groups appended to an alkyl group. The term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to five groups independently selected from, for example, halogen, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

The term “heteroaryl” refers to a monocyclic or polycyclic aromatic group, preferably a monocyclic or bicyclic aromatic group, containing at least one nitrogen, oxygen, or sulfur atom in an aromatic ring. Unless otherwise indicated, a heteroaryl group can be unsubstituted or substituted with 1 to 5 groups. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

The term “alkylaryl” refers to one or more alkyl groups appended to an aryl group.

The term “alkoxyaryl” refers to one or more alkoxy groups appended to an aryl group.

The term “arylalkyl” refers to one or more aryl groups appended to an alkyl group.

The term “aryloxy” refers to an aromatic group covalently bonded to the parent molecule through an —O— linkage.

The term “alkylaryloxy” refers to an alkylaryl group covalently bonded to the parent molecule through an —O—linkage.

The term “alkanol” refers to a straight or branched chain alkyl group covalently bonded to OH.

The term “alkanolamine” refers to straight or branched chain alkyl groups covalently bonded to a hydroxy moiety and to a amino moiety. Examples of alkanolamine include propanolamine, ethanolamine, dimethylethanolamine, and the like.

The term “amido” refers to a group having a NH₂ radical that is bonded to a C═O radical.

The term “alkanolamide” refers to a straight or branched chain alkyl group covalently bonded to a hydroxy moiety and to an amide moiety.

The term “alkylsulfate” refers to a straight or branched chain alkyl group covalently to SO₃⁻.

The term “benzyl” refers to a benzene radical that can be unsubstituted or substituted with one or more, and in particular one to five groups independently selected from, for example, halogen, alkyl, alkenyl, OCF₃, NO₂, CN, NC, OH, alkoxy, amino, CO₂H, CO₂alkyl, aryl, and heteroaryl.

The term “halogen” or “halo” refers to fluoro, chloro, bromo, or iodo.

All percentages, parts and ratios are based upon the total weight of the compositions of which the ingredient is a part, unless otherwise specified. All such weights as they pertain to listed ingredients are based on the active level and therefore do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

Anionic Surfactant System—Ethoxylate Level and Anion Level

The personal care compositions prepared in accordance with the inventive methods disclosed herein include an anionic surfactant system. The anionic surfactant system is included to provide cleaning performance to the composition. The anionic surfactant system includes at least one anionic surfactant, and optionally an amphoteric surfactant, a zwitterionic surfactant, a cationic surfactant, a nonionic surfactant, or a combination thereof. Such surfactants should be physically and chemically compatible with other components (ingredients) described herein, or should not otherwise unduly impair product stability, aesthetics, or performance.

Suitable anionic surfactant components for use in personal care compositions include those that are known for use in hair care or other personal care compositions. The concentration of the anionic surfactant system in these compositions should be sufficient to provide the desired cleaning and lather performance, and generally ranges from about 5% to about 50%, in one embodiment from about 8% to about 30%, in another embodiment from about 10% to about 25%, by weight of the composition.

In considering the performance characteristics of a personal care composition, such as coacervate formation, wet conditioning performance, dry conditioning performance, and conditioning ingredient deposition on hair, the levels and types of surfactants are desirably optimized to maximize the performance potential of polymer systems. The anionic surfactant system for use in personal care compositions has an ethoxylate level and an anion level, wherein the ethoxylate level is from about 1 to about 6, and wherein the anion level is from about 1 to about 6. The combination of such an anionic surfactant system with the cationically modified starch polymers of the present personal care compositions provide enhanced deposition of conditioning agents to hair and/or skin without reducing cleansing performance.

An optimal ethoxylate level is calculated based on the stoichiometry of the surfactant structure, which in turn is based on a particular molecular weight of the surfactant where the number of moles of ethoxylation is known. Likewise, given a specific molecular weight of a surfactant and an anionization reaction completion measurement, the anion level can be calculated. Analytical techniques have been developed to measure ethoxylation or anionization within surfactant systems.

The Level of Ethoxylate and the Level of Anion representative of a particular surfactant system are calculated from the percent ethoxylation and percent anion of individual surfactants in the following manner. The Level of Ethoxylate is equal to the percent ethoxylation multiplied by percent active ethoxylated surfactant (based on the total weight of the composition). The Level of Anion is equal to the percent anion in ethoxylated surfactant multiplied by percent active ethoxylated surfactant (based on the total weight of the composition) plus percent anion in non-ethoxylated surfactant (based on the total weight of the composition). If a composition comprises two or more surfactants having different respective anions (e.g., surfactant A has a sulfate group and surfactant B has a sulfonate group), the Level of Anion in the composition is the sum of the molar levels of each respective anion as calculated above.

For example, a detersive surfactant contains 48.27 wt. % sodium laureth sulfate 3-ethoxylate (SLE3S) and 6.97 wt. % sodium lauryl sulfate (SLS), based on the total weight of the composition. The ethoxylated surfactant (SLE3S) contains 0.294321% ethoxylate and 0.188307% sulfate as the anion and the non-ethoxylated surfactant (SLS) contains 0.266845% sulfate as an anion. Because both of the SLE and SLS are about 29% active, the detersive surfactant contains about 14 wt. % of active SLE3S and about 2 wt. % of active SLS, based on the total weight of the composition. The Level of Ethoxylate is 0.294321 multiplied by 14 (% active ethoxylated surfactant). Thus, the Level of Ethoxylate in this example detersive surfactant is 4.12. The Level of Anion is 0.188307 multiplied by 14 (% active ethoxylated surfactant) plus 0.266845 multiplied by 2 (% active non-ethoxylated surfactant). Thus the Level of Anion in this example detersive surfactant is 3.17.

In one embodiment, the detersive surfactant includes at least one anionic surfactant that contains an anion selected from the group consisting of sulfates, sulfonates, sulfosuccinates, isethionates, carboxylates, phosphates, and phosphonates. In another embodiment, the anion is a sulfate. Other potential anions for the anionic surfactant include phosphonates, phosphates, and carboxylates.

Anionic surfactants suitable for use in the personal care compositions are alkyl sulfates and alkyl ether sulfates. These materials have the respective formulae ROSO₃M and RO(C₂H₄O)ₓSO₃M, wherein R is alkyl or alkenyl of about 8 to about 18 carbon atoms, x is an integer having a value of about 1 to about 10, and M is a cation such as ammonium, an alkanolamine such as triethanolamine, a monovalent metal cation such as sodium and potassium, or a polyvalent metal cation such as magnesium and calcium. Solubility of the surfactant will depend upon the particular anionic surfactants and cations chosen.

In one embodiment, R has about 8 to about 18 carbon atoms, in another embodiment about 10 to about 16 carbon atoms, and in yet another embodiment from about 12 to about 14 carbon atoms, in both the alkyl sulfates and alkyl ether sulfates. The alkyl ether sulfates are typically made as condensation products of ethylene oxide and monohydric alcohols having about 8 to about 24 carbon atoms. The alcohols can be synthetic or they can be derived from fats, e.g., coconut oil, palm kernel oil, tallow. In one embodiment the lauryl alcohol and straight chain alcohols are derived from coconut oil or palm kernel oil. Such alcohols are reacted with about 0 to about 10, in one embodiment from about 0 to about 5, in another embodiment from about 0, 1 or 3, molar proportions of ethylene oxide, and the resulting mixture of molecular species having, for example, an average of 0, 1, or 3 moles of ethylene oxide per mole of alcohol is sulfated and neutralized.

Specific non-limiting examples of alkyl ether sulfates that may be used in personal care composition include sodium and ammonium salts of coconut alkyl triethylene glycol ether sulfate, tallow alkyl triethylene glycol ether sulfate, and tallow alkyl hexa-oxyethylene sulfate. In one embodiment the alkyl ether sulfates are those that include a mixture of individual compounds, wherein the compounds in the mixture have an average alkyl chain length of from about 10 to about 16 carbon atoms and an average degree of ethoxylation of from about 1 to about 4 moles of ethylene oxide. Such a mixture also includes from about 0% to about 20% by weight C₁₂₋₁₃ compounds; from about 60% to about 100% by weight of C₁₄₋₁₅₋₁₆ compounds; from about 0% to about 20% by weight of C₁₇₋₁₈₋₁₉ compounds; from about 3% to about 30% by weight of compounds having a degree of ethoxylation of 0; from about 45% to about 90% by weight of compounds having a degree of ethoxylation from about 1 to about 4; from about 10% to about 25% by weight of compounds having a degree of ethoxylation from about 4 to about 8; and from about 0.1% to about 15% by weight of compounds having a degree of ethoxylation greater than about 8, based on the total weight of the alkyl ether sulfate.

Suitable anionic detersive surfactant components include those which are known for use in hair care or other personal care cleansing compositions. In one embodiment the anionic detersive surfactants components include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

In some embodiments, the detersive surfactant further includes one or more additional surfactants selected from the group consisting of amphoteric surfactants, Zwitterionic surfactants, cationic surfactants, nonionic surfactants, and mixtures thereof. These surfactants are known for use in hair care or other personal care cleansing compositions and contain a group that is anionic at the pH of the composition. The concentration of such amphoteric detersive surfactants in one embodiment ranges from about 0.5 wt. % to about 20 wt. %, in another embodiment from about 1 wt. % to about 10 wt. %, based on the total weight of the detersive surfactant. Non-limiting examples of suitable Zwitterionic or amphoteric surfactants are described in U.S. Pat. Nos. 5,104,646 and 5,106,609.

Suitable amphoteric surfactants are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Suitable amphoteric surfactants for use in the personal care compositions further include cocoamphoacetate, cocoamphodiacetate, lauroamphoacetate, lauroamphodiacetate, lauramine oxide, and mixtures thereof.

Zwitterionic surfactants suitable for use in the personal care composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In one embodiment zwitterionic surfactant is a such as betaine (i.e., cocoamidopropyl betaine, cocobetaine) are suitable for use.

Additional surfactants may be used in combination with the detersive surfactant component described herein. Other suitable anionic surfactants are the water-soluble salts of organic, sulfuric acid reaction products conforming to the formula [R¹—SO₃-M] where R¹ is a straight or branched chain, saturated, aliphatic hydrocarbon radical having from about 8 to about 24, and in one embodiment from about 10 to about 18 carbon atoms; and M is a cation, as previously described herein. Nonlimiting examples of such surfactants are the salts of an organic sulfuric acid reaction product of a hydrocarbon of the methane series, including iso-, neo-, and n-paraffins, having from about 8 to about 24 carbon atoms, in one embodiment about 12 to about 18 carbon atoms and a sulfonating agent, e.g., SO₃, H₂SO₄, obtained according to known sulfonation methods, including bleaching and hydrolysis. In one embodiment alkali metal and ammonium sulfonated C₁₀₋₁₈ n-paraffins are suitable for use.

Other suitable anionic surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil.

Other anionic surfactants suitable for use in the composition of the invention are the succinates, examples of which include disodium N-octadecylsulfosuccinate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having about 10 to about 24 carbon atoms. In this context, the term “olefin sulfonates” refers to compounds which can be produced by the sulfonation of alpha-olefins by means of uncomplexed sulfur trioxide, followed by neutralization of the acid reaction mixture in conditions such that any sulfonates which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form. The alpha-olefins from which the olefin sulfonates are derived are mono-olefins having about 10 to about 24 carbon atoms, in one embodiment about 12 to about 16 carbon atoms. In another embodiment, they are straight chain olefins. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A nonlimiting example of such an alpha-olefin sulfonate mixture is described in U.S. Pat. No. 3,332,880.

Another class of anionic surfactants suitable for use in the compositions of the invention are the beta-alkyloxy alkane sulfonates. These surfactants conform to the Formula I:

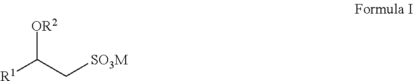

where R¹ is a straight chain alkyl group having about 6 to about 20 carbon atoms, R² is a lower alkyl group having about 1 to about 3 carbon atoms, and in one embodiment 1 carbon atom, and M is a water-soluble cation, as previously described herein. Suitable anionic surfactants for use in the composition of the invention include sodium tridecyl benzene sulfonate, sodium dodecyl benzene sulfonate, and mixtures thereof.

Amides, including alkanolamides, are the condensation products of fatty acids with primary and secondary amines or alkanolamines to yield products of the general Formula II:

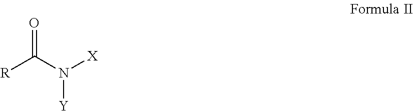

wherein RCO is a fatty acid radical and R is C₈₋₂₀; X is an alkyl, aromatic or alkanol (CHR¹CH₂OH wherein R′ is H or C₁₋₆ alkyl); Y is H, alkyl, alkanol or X. Suitable amides include, but are not limited to cocamide, lauramide, oleamide and stearamide. Suitable alkanolamides include, but are not limited to, cocamide DEA, cocamide MEA, cocamide MIPA, isostearamide DEA, isostearamide MEA, isostearamide MIPA, lanolinamide DEA, lauramide DEA, lauramide MEA, lauramide MIPA, linoleamide DEA, linoleamide MEA, linoleamide MIPA, myristamide DEA, myristamide MEA, myristamide MIPA, Oleamide DEA, Oleamide MEA, Oleamide MIPA, palmamide DEA, palmamide MEA, palmamide MIPA, palmitamide DEA, palmitamide MEA, palm kernelamide DEA, palm kernelamide MEA, palm kernelamide MIPA, peanutamide MEA, peanutamide MIPA, soyamide DEA, stearamide DEA, stearamide MEA, stearamide MIPA, tallamide DEA, tallowamide DEA, tallowamide MEA, undecylenamide DEA, undecylenamide MEA, PPG-2 Hydroxyethyl cocoamide, and PPG-2-Hydroxyethyl Coco/Isostearamide. The condensation reaction may be carried out with free fatty acids or with all types of esters of the fatty acids, such as fats and oils, and particularly methyl esters. The reaction conditions and the raw material sources determine the blend of materials in the end product and the nature of any impurities.

Suitable optional surfactants include nonionic surfactants. Any such surfactant known in the art for use in hair or personal care products may be used, provided that the optional additional surfactant is also chemically and physically compatible with the essential components of the composition of the invention, or does not otherwise unduly impair product performance, aesthetics or stability. The concentration of the optional additional surfactants in the personal care composition may vary with the cleansing or lather performance desired, the optional surfactant selected, the desired product concentration, the presence of other components in the composition, and other factors well known in the art.

Nonlimiting examples of other surfactants suitable for use in the personal care compositions are described in McCutcheon's, Emulsifiers and Detergents, 1989 Annual, published by M.C. Publishing Co., and U.S. Pat. Nos. 3,929,678; 2,658,072; 2,438,091; 2,528,378.

Viscosity Modifiers

Hydrotropes

Suitable hydrotropes that may be used in accordance with embodiments of the invention include short chain surfactants that help solubilize surfactants. In some embodiments, the hydrotrope includes C₁₋₈alkyl carboxylates, C₁₋₈alkyl sulfates, C₁₋₈alkyl benzene sulfonates, halogen benzoates (e.g., chlorbenzoate), C₁₋₈alkyl naphthalene carboxylates, (e.g., hydroxyl naphthalene carboxylate), urea, ethoxylated sulfates, and mixtures thereof. The C₁₋₈alkyl benzene sulfonates can include C₁₋₈alkyl cumene sulfonates, C₁₋₈alkyl toluene sulfonates (e.g., para-toluene sulfonate), C₁-C₈alkyl xylene sulfonates, and mixtures thereof. For example, the hydrotrope can include, sodium xylene sulfonates, potassium xylene sulfonates, ammonium xylene sulfonates, calcium xylene sulfonates, sodium toluene sulfonates, potassium toluene sulfonates, sodium cumene sulfonates, ammonium cumene sulfonates, sodium alkyl naphthalene sulfonates, sodium butyl naphthalene sulfonates, and mixtures thereof, many of which are commercially available from Nease Corporation (Cincinnati, Ohio).

Electrolytes

A suitable viscosity modifier, one that may result in an increase in viscosity, is a compound that liberates a cation in the presence of the ionic surfactant-containing feed. In one embodiment, the cation is selected from the group consisting of calcium, potassium, sodium, lithium, ammonium, and tetraethyl ammonium (TEA). Examples of such compounds include chlorides and bromides of calcium, potassium, sodium, lithium, ammonium, and TEA.

Nonlimiting examples of inorganic salts suitable for use in the composition of the invention include MgI₂, MgBr₂, MgCl₂, Mg(NO₃)₂, Mg₃(PO₄)₂, Mg₂P₂O₇, MgSO₄, magnesium silicate, NaI, NaBr, NaCl, NaF, Na₃(PO₄), NaSO₃, Na₂SO₄, Na₂SO₃, NaNO₃, NaIO₃, Na₃(PO₄), Na₄P₂O₇, sodium silicate, sodium metasilicate, sodium tetrachloroaluminate, sodium tripolyphosphate (STPP), Na₂Si₃O₇, sodium zirconate, CaF₂, CaCl₂, CaBr₂, CaI₂, CaSO₄, Ca(NO₃)₂, Ca, KI, KBr, KCl, KF, KNO₃, KIO₃, K₂SO₄, K₂SO₃, K₃(PO₄), K₄(P₂O₇), potassium pyrosulfate, potassium pyrosulfite, LiI, LiBr, LiCl, LiF, LiNO₃, AlF₃, AlCl₃, AlBr₃, AlI₃, Al₂(SO₄)₃, Al(PO₄), A(NO₃)₃, aluminum silicate; including hydrates of these salts and including combinations of these salts or salts with mixed cations e.g. potassium alum AlK(SO₄)₂ and salts with mixed anions, e.g. potassium tetrachloroaluminate and sodium tetrafluoroaluminate. Mixtures of above salts are also useful

Organic salts useful in this invention include, magnesium, sodium, lithium, potassium, zinc, and aluminum salts of the carboxylic acids including formate, acetate, proprionate, pelargonate, citrate, gluconate, lactate aromatic acids e.g., benzoates, phenolate and substituted benzoates or phenolates, such as phenolate, salicylate, polyaromatic acids terephthalates, and polyacids e.g., oxylate, adipate, succinate, benzenedicarboxylate, benzenetricarboxylate. Other useful organic salts include carbonate and/or hydrogencarbonate (HCO₃⁻¹) when the pH is suitable, alkyl and aromatic sulfates and sulfonates e.g., sodium methyl sulfate, benzene sulfonates and derivatives such as xylene sulfonate, and amino acids when the pH is suitable. Electrolytes can comprise mixed salts of the above, salts neutralized with mixed cations such as potassium/sodium tartrate, partially neutralized salts such as sodium hydrogen tartrate or potassium hydrogen phthalate, and salts comprising one cation with mixed anions.

### Polymeric Thickeners

Personal care compositions can include one or more polymeric thickening agents, in one embodiment from about 0.1% to about 5%, in another embodiment from about 0.1% to about 3%, and in yet another embodiment from about 0.25% to about 2%, by weight of the composition. Polymeric thickening agents suitable for use herein include those selected from the group consisting of carboxylic acid polymers, crosslinked polyacrylate polymers, polyacrylamide polymers, and mixtures thereof, and in another embodiment from the group consisting of carboxylic acid polymers, polyacrylamide polymers, and mixtures thereof.

Carboxylic acid polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present composition are more fully described in U.S. Pat. Nos. 5,087,445, 4,509,949, and 2,798,053, and in CTFA International Cosmetic Ingredient Dictionary, Fourth edition, 1991, pp. 12 and 80.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, Pemulen TR-1, and Pemulen TR-2, from B.F. Goodrich. In other words, examples of carboxylic acid polymer thickeners useful herein are those selected from the group consisting of carbomers, acrylates/C₁₀₋₃₀ alkyl acrylate crosspolymers, and mixtures thereof.

Crosslinked polyacrylate polymers include both cationic and nonionic polymers. In one embodiment the polymer is cationic. Examples of useful crosslinked nonionic polyacrylate polymers and crosslinked cationic polyacrylate polymers are those described in U.S. Pat. Nos. 5,100,660, 4,849,484, 4,835,206, 4,628,078, and 4,599,379, and European patent publication No. EP 228,868.

In one embodiment the polyacrylamide polymers are nonionic polyacrylamide polymers including substituted branched or unbranched polymers. In another embodiment the polyacrylamide polymers is the nonionic polymer given the CTFA designation polyacrylamide and isoparaffin and laureth-7, available under the Tradename Sepigel 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS50OW, SSSA100H, from Lipo Chemicals, Inc., (Patterson, N.J.).

A wide variety of polysaccharides are useful herein. “Polysaccharides” refer to gelling agents that contain a backbone of repeating sugar (i.e. carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers, the hydroxy groups of the cellulose polymer is hydroxyalkylated (including, but not limited to hydroxyethylated or hydroxypropylated) to form a hydroxyalkylated cellulose which is then further modified with a C₁₀₋₃₀ straight chain or branched chain alkyl group through an ether linkage. Typically these polymers are ethers of C₁₀₋₃₀ straight or branched chain alcohols with hydroxyalkylcelluloses. Examples of alkyl groups useful herein include those selected from the group consisting of stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. In one embodiment the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation (Wilmington, Del.). Other useful polysaccharides include scleroglucans comprising a linear chain of (1-3) linked glucose units with a (1-6) linked glucose every three units, a commercially available example of which is Clearogel™ CS11 from Michel Mercier Products Inc. (Mountainside, N.J.).

Other thickening and gelling agents useful herein include materials that are primarily derived from natural sources. Nonlimiting examples of these gelling agent gums include materials selected from the group consisting of acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

Additional components of personal care compositions that may be made in accordance with embodiments of the inventive methods disclosed herein are set out below.

### 1. Conditioning Agents

a. Oily Conditioning Agent

In certain embodiments, personal care compositions can include one or more oily conditioning agents. Oily conditioning agents include materials that are used to give a particular conditioning benefit to hair and/or skin. In hair treatment compositions, suitable conditioning agents are those that deliver one or more benefits relating to shine, softness, combability, antistatic properties, wet-handling, damage, manageability, body, and greasiness. The oily conditioning agents useful in these compositions typically include a water-insoluble, water-dispersible, non-volatile, liquid that forms emulsified, liquid particles. Suitable oily conditioning agents are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters,) or combinations thereof, or those conditioning agents that otherwise form liquid, dispersed particles in the aqueous surfactant matrix herein.

One or more oily conditioning agents are typically present in one embodiment at a concentration of from about 0.01 wt. % to about 10 wt. %, in another embodiment from about 0.1 wt. % to about 8 wt. %, in yet another embodiment from about 0.2 wt. % to about 4 wt. %, based on the weight of the personal care composition.

b. Silicone Conditioning Agent

In one embodiment the oily conditioning agents of the compositions are a water-insoluble silicone conditioning agent. The silicone conditioning agent may comprise volatile silicone, non-volatile silicone, or combinations thereof. Suitable for use herein are non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. In one embodiment silicone conditioning agents for use in the personal care compositions have a viscosity, as measured at 25° C. of in one embodiment from about 20 to about 2,000,000 centistokes (“csk”), in another embodiment from about 1,000 to about 1,800,000 csk, in yet another embodiment from about 5,000 to about 1,500,000 csk, and in yet another embodiment from about 10,000 to about 1,000,000 csk.

In embodiments of personal care compositions that are opaque, a non-volatile silicone oil having a particle size as measured in the personal care composition from about 1 micrometers (μm) to about 50 μm may be included. In embodiments for small particle application to hair, the personal care composition can include a non-volatile silicone oil having a particle size as measured in the personal care composition from about 100 nanometers (nm) to about 1 μm. A substantially clear composition embodiment of personal care compositions includes a non-volatile silicone oil having a particle size as measured in the personal care composition of less than about 100 nm.

Suitable non-volatile silicone oils may be selected from organo-modified silicones and fluoro-modified silicones. The non-volatile silicone oil may be an organo-modified silicone that includes an organo group selected from the group consisting of alkyl groups, alkenyl groups, hydroxyl groups, amine groups, quaternary groups, carboxyl groups, fatty acid groups, ether groups, ester groups, mercapto groups, sulfate groups, sulfonate groups, phosphate groups, propylene oxide groups, and ethylene oxide groups. In one embodiment the non-volatile silicone oil is dimethicone.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

Silicone fluids generally suitable for use in personal care compositions disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984).

c. Organic Conditioning Oils

The oily conditioning agent of the personal care compositions can further include at least one organic conditioning oil, either alone or in combination with other conditioning agents, such as the silicones described above.

d. Hydrocarbon Oils

Suitable organic conditioning oils for use as conditioning agents in the personal care compositions include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils can be from about C₁₂ to about C₁₉. Branched chain hydrocarbon oils, including hydrocarbon polymers, typically will contain more than 19 carbon atoms.

Specific nonlimiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include 2,2,4,4,6,6,8,8-dimethyl-10-methylundecane and 2,2,4,4,6,6-dimethyl-8-methylnonane, available from Permethyl Corporation. In one embodiment the hydrocarbon polymer is polybutene, such as the copolymer of isobutylene and butene, which is commercially available as L-14 polybutene from Amoco Chemical Corporation.

e. Polyolefins

Organic conditioning oils for use in the personal care compositions can also include liquid polyolefins, including but not limited toliquid poly-α-olefins, and hydrogenated liquid poly-α-olefins. Polyolefins for use herein are prepared by polymerization of C₄ to about C₁₄ olefenic monomers in one embodiment, and from about C₆ to about C₁₂ in another embodiment.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents.

f. Fatty Esters

Other suitable organic conditioning oils for use as the conditioning agent in the personal care compositions include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

Examples of suitable fatty esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate. Other fatty esters suitable for use in the compositions hereinare those known as polyhydric alcohol esters. Such polyhydric alcohol esters include alkylene glycol esters.

Still other fatty esters suitable for use in the personal care compositions are glycerides, including, but not limited to, mono-, di-, and tri-glycerides. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

g. Fluorinated Conditioning Compounds

Fluorinated compounds suitable for delivering conditioning to hair or skin as organic conditioning oils include perfluoropolyethers, perfluorinated olefins, fluorine based specialty polymers that may be in a fluid or elastomer form similar to the silicone fluids previously described, and perfluorinated dimethicones. Specific non-limiting examples of suitable fluorinated compounds include the Fomblin product line from Ausimont which includes HC/04, HC/25, HC01, HC/02, HC/03; Dioctyldodecyl Fluoroeptyl Citrate, commonly called Biosil Basics Fluoro Gerbet 3.5 supplied by Biosil Technologies; and Biosil Basics Fluorosil LF also supplied by Biosil Technologies.

h. Fatty Alcohols

Other suitable organic conditioning oils for use in the personal care compositions include, but are not limited to, fatty alcohols having at least about 10 carbon atoms, and in one embodiment from about 10 to about 22 carbon atoms, in another embodiment from about 12 to about 16 carbon atoms. Also suitable for use in the personal care compositions herein are alkoxylated fatty alcohols which conform to the general formula:

CH₃(CH₂)ₙCH₂(OCH₂CH₂)ₚOH

wherein n is a positive integer having a value from about 8 to about 20, in one embodiment from about 10 to about 14, and p is a positive integer having a value from about 1 to about 30, and in one embodiment from about 2 to about 23.

i. Alkyl Glucosides and Alkyl Glucoside Derivatives

Suitable organic conditioning oils for use in the personal care compositions include, but are not limited to, alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.

Other Conditioning Agents

j. Quaternary Ammonium Compounds

Suitable quaternary ammonium compounds for use as conditioning agents in the personal care compositions include, but are not limited to, hydrophilic quaternary ammonium compounds with a long chain substituent having a carbonyl moiety, like an amide moiety, or a phosphate ester moiety or a similar hydrophilic moiety.

Examples of useful hydrophilic quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

Examples of other useful quaternary ammonium surfactants include, but are not limited to, Quaternium-33, Quaternium-43, isostearamidopropyl ethyldimonium ethosulfate, Quaternium-22 and Quaternium-26, or combinations thereof, as designated in the CTFA Dictionary.

Other hydrophilic quaternary ammonium compounds useful in the present composition include, but are not limited to, Quaternium-16, Quaternium-27, Quaternium-30, Quaternium-52, Quaternium-53, Quaternium-56, Quaternium-60, Quatemium-61, Quaternium-62, Quaternium-63, Quatemium-71, and combinations thereof.

k. Polyethylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M and mixtures thereof. Glycerin may also be used as conditioning agent in the personal care compositions. In one embodiment, glycerin may be present in a range of about 0.01 wt. % to about 10 wt. %, based on the total weight of the personal care product. In a further embodiment, glycerin may be present in a range of about 0.1 wt. % to about 5 wt. %, based on the total weight of the personal care product. In yet a further embodiment, glycerin may be present in a range of about 2 wt. % to about 4 wt. %, based on the total weight of the personal care product.

### 2. Additional Components

The personal care compositions that may be made in accordance with the inventive methods disclosed herein may include one or more additional components known for use in hair care or personal care products, provided that the additional components are physically and chemically compatible with the essential components described herein, or do not otherwise unduly impair product stability, aesthetics or performance. Individual concentrations of such additional components may range from about 0.001% to about 10% by weight of the personal care compositions.

Non-limiting examples of additional components for use in the composition include natural cationic deposition polymers, synthetic cationic deposition polymers, anti-dandruff agents, particles, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins.

a. Cellulose or Guar Cationic Deposition Polymers

The personal care compositions may also include cellulose or guar cationic deposition polymers. In one embodiment the composition comprises a cellulose or glactomannan cationic deposition polymers. Generally, such cellulose or guar cationic deposition polymers may be present at a concentration from about 0.05% to about 5%, by weight of the composition. Suitable cellulose or guar cationic deposition polymers have a molecular weight of greater than about 5,000. In one embodiment the cellulose or guar cationic deposition polymers has a molecular weight of greater than about 200,000. Additionally, such cellulose or guar deposition polymers have a charge density from about 0.15 milliequivalents per gram (meq/g) to about 4.0 meq/g at the pH of intended use of the personal care composition, which pH will generally range from about pH 3 to about pH 9, and in one embodiment between about pH 4 and about pH 8. The pH of the personal care compositions are measured neat.

Suitable cellulose or guar cationic polymers include, but are not limited to, those which conform to the following formula:

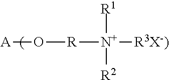

wherein A is an anhydroglucose residual group, such as a cellulose anhydroglucose residual; R is an alkylene oxyalkylene, polyoxyalkylene, or hydroxyalkylene group, or combination thereof, R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) being about 20 or less; and X is an anionic counterion. Non-limiting examples of such counterions include halides (e.g., chlorine, fluorine, bromine, iodine), sulfate and methylsulfate. The degree of cationic substitution in these polysaccharide polymers is typically from about 0.01 to about 1 cationic groups per anhydroglucose unit.

In one embodiment, the cellulose or guar cationic polymers are salts of hydroxyethyl cellulose reacted with trimethyl ammonium substituted epoxide, referred to in the industry (CTFA) as Polyquaternium 10 and available from Amerchol Corp. (Edison, N.J., USA).

Other suitable cationic deposition polymers include cationic guar gum derivatives, such as guar hydroxypropyltrimonium chloride, specific examples of which include the Jaguar series (in one embodiment Jaguar C-17®) commercially available from Rhone-Poulenc Incorporated, and further including Jaguar C-500, commercially available from Rhodia.

b. Synthetic Cationic Deposition Polymers

The personal care compositions may also include synthetic cationic deposition polymers. Generally, such synthetic cationic deposition polymers may be present at a concentration from about 0.025% to about 5%, by weight of the composition. Such synthetic cationic deposition polymers have a molecular weight from about 1,000 to about 5,000,000. Additionally, such synthetic cationic deposition polymers have a charge density from about 0.1 meq/g to about 5.0 meq/g.

Suitable synthetic cationic deposition polymers include those that are water-soluble or dispersible, cationic, non-crosslinked, conditioning copolymers comprising: (i) one or more cationic monomer units; and (ii) one or more nonionic monomer units or monomer units bearing a terminal negative charge; wherein said copolymer has a net positive charge, a cationic charge density of from about 0.5 meq/g to about 10 meg/g, and an average molecular weight from about 1,000 to about 5,000,000. Non-limiting examples of suitable synthetic cationic deposition polymers are described in US 2003/0223951 A1.

Anti-Dandruff Actives

Personal care compositions such as shampoo may also contain an anti-dandruff agent. Suitable, non-limiting examples of anti-dandruff particulates include: pyridinethione salts, zinc carbonate, azoles, such as ketoconazole, econazole, and elubiol, selenium sulfide, particulate sulfur, salicylic acid and mixtures thereof. A typical anti-dandruff particulate is pyridinethione salt. Such anti-dandruff particulate should be physically and chemically compatible with the components of the composition, and should not otherwise unduly impair product stability, aesthetics or performance.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Azole anti-microbials include imidazoles such as climbazole and ketoconazole.

Selenium sulfide compounds are described, for example, in U.S. Pat. No. 2,694,668; U.S. Pat. No. 3,152,046; U.S. Pat. No. 4,089,945; and U.S. Pat. No. 4,885,107.

Sulfur may also be used as a particulate anti-microbial/anti-dandruff agent in the anti-microbial compositions.

Personal care compositions may further include one or more keratolytic agents such as Salicylic Acid.

Additional anti-microbial actives may include extracts of melaleuca (tea tree) and charcoal.

When present in personal care compositions, the anti-dandruff active is included in an amount of from about 0.01 wt. % to about 5 wt. %, in one embodiment from about 0.1 wt. % to about 3 wt. %, and in yet another embodiment about 0.3 wt. % to about 2 wt. %, based on the weight of the personal care product.

d. Particles

The personal care compositions optionally may include particles. Suitable for use herein are particles dispersed water-insoluble particles, and can be inorganic, synthetic, or semi-synthetic. In one embodiment no more than 20% of particles are incorporated in another embodiment no more than about 10% and in yet another embodiment no more than 2%, by weight of the composition, of particles. In certain embodiments, the particles have an average mean particle size of less than about 300 μm.

Non-limiting examples of inorganic particles include colloidal silicas, fumed silicas, precipitated silicas, silica gels, magnesium silicate, glass particles, talcs, micas, sericites, and various natural and synthetic clays including bentonites, hectorites, and montmorillonites. Examples of synthetic particles include silicone resins, poly(meth)acrylates, polyethylene, polyester, polypropylene, polystyrene, polyurethane, polyamide (e.g., Nylon®), epoxy resins, urea resins, acrylic powders, and the like. Non-limiting examples of hybrid particles include sericite & crosslinked polystyrene hybrid powder, and mica and silica hybrid powder.

e. Opacifying Agents

Personal care compositions may also contain one or more opacifying agents. Opacifying agents are typically used in cleansing compositions to impart desired aesthetic benefits to the composition, such as color or pearlescence. In one embodiment the opacifying agents are included at no more than about 20%, in another embodiment at no more than about 10% and in yet another embodiment at no more than 2%, by weight of the composition, of opacifying agents.

Suitable opacifying agents include, for example, fumed silica, polymethylmethacrylate, micronized Teflon®, boron nitride, barium sulfate, acrylate polymers, aluminum silicate, aluminum starch octenylsuccinate, calcium silicate, cellulose, chalk, corn starch, diatomaceous earth, Fuller's earth, glyceryl starch, hydrated silica, magnesium carbonate, magnesium hydroxide, magnesium oxide, magnesium trisilicate, maltodextrin, microcrystalline cellulose, rice starch, silica, titanium dioxide, zinc laurate, zinc myristate, zinc neodecanoate, zinc rosinate, zinc stearate, polyethylene, alumina, attapulgite, calcium carbonate, calcium silicate, dextran, nylon, silica silylate, silk powder, soy flour, tin oxide, titanium hydroxide, trimagnesium phosphate, walnut shell powder, or mixtures thereof. The above mentioned powders may be surface treated with lecithin, amino acids, mineral oil, silicone oil, or various other agents either alone or in combination, which coat the powder surface and render the particles hydrophobic in nature.

The opacifying agents may also include various organic and inorganic pigments. The organic pigments are generally various aromatic types including azo, indigoid, triphenylmethane, anthraquinone, and xanthine dyes. Inorganic pigments include iron oxides, ultramarine and chromium or chromium hydroxide colors, and mixtures thereof.

f. Suspending Agents

Personal care compositions may further include a suspending agent at concentrations effective for suspending water-insoluble material in dispersed form in the compositions or for modifying the viscosity of the composition. Such concentrations generally range from about 0.1% to about 10%, and in one embodiment from about 0.3% to about 5.0%, by weight of the composition, of suspending agent.

Suspending agents useful herein include anionic polymers and nonionic polymers. Useful herein are vinyl polymers such as cross linked acrylic acid polymers with the CTFA name Carbomer.

Other optional suspending agents include crystalline suspending agents, which can be categorized as acyl derivatives, long chain amine oxides, and mixtures thereof. These suspending agents are described in U.S. Pat. No. 4,741,855. These suspending agents may include ethylene glycol esters of fatty acids having from about 16 to about 22 carbon atoms. In another embodiment the suspending agents are the ethylene glycol stearates, both mono and distearate, but particularly the distearate containing less than about 7% of the mono stearate.

Other suitable suspending agents include alkanol amides of fatty acids, in one embodiment having from about 16 to about 22 carbon atoms, in another embodiment having about 16 to 18 carbon atoms, suitable examples of which include stearic monoethanolamide, stearic diethanolamide, stearic monoisopropanolamide and stearic monoethanolamide stearate.

Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Long chain acyl derivatives, ethylene glycol esters of long chain carboxylic acids, long chain amine oxides, and alkanol amides of long chain carboxylic acids.

g. Paraffinic Hydrocarbons

Personal care compositions may contain one or more paraffinic hydrocarbons. Suitable paraffinic hydrocarbons include those materials that are known for use in hair care or other personal care compositions, such as those having a vapor pressure at 1 atm of equal to or greater than about 21° C. (about 70° F.). Non-limiting examples include pentane and isopentane.

h. Propellants

Personal care compositions also may contain one or more propellants, suitable examples of which include those materials that are known for use in hair care or other personal care compositions, such as liquefied gas propellants and compressed gas propellants. Suitable propellants have a vapor pressure at 1 atm of less than about 21° C. (about 70° F.). Non-limiting examples of suitable propellants are alkanes, isoalkanes, haloalkanes, dimethyl ether, nitrogen, nitrous oxide, carbon dioxide, and mixtures thereof.

Other Optional Components

Personal care compositions may contain one or more fragrances. The fragrances are used for aesthetic purposes and can be present in an amount of about 0.25 wt. % to about 2.5 wt. %, based on the total weight of the composition.

Personal care compositions may also contain water-soluble and water-insoluble vitamins such as vitamins B1, B2, B6, B12, C, pantothenic acid, pantothenyl ethyl ether, panthenol, biotin and their derivatives, and vitamins A, D, E, and their derivatives. The compositions may also contain water-soluble and water-insoluble amino acids such as asparagine, alanine, indole, glutamic acid and their salts, and tyrosine, tryptamine, lysine, histadine and their salts.

The compositions may also contain chelating agents. The chelating agent functions to potentiate preservatives and is present in an active amount of up to about 0.5 wt. %, based on the total weight of the personal care product.

The compositions may further include materials useful for hair loss prevention and hair growth stimulants or agents.

### Example

The following example is presented to demonstrate an application of the inventive methods in the context of the preparation of a shampoo formulation. The application of these methods, however, is not limited to the manufacture of a shampoo formulation; but, instead, can be employed in the manufacture of many other personal care compositions, as well as laundry detergent compositions and cleaning compositions.

Determining the Ingredients that will Influence Viscosity

It was determined that a shampoo formulation that includes 65 wt. % of a V-chassis Vanilla Base, based on the total weight composition, would be made. The V-chassis Vanilla Base would include the materials specified in Table 1a, below:

TABLE 1a

| V-chassis Vanilla | |
|---|---|
| Base Ingredients | Wt. % |
| Water | 42.1 |
| Citric Acid | 0.3 |
| EDTA | 0.2 |
| Sodium Benzoate | 0.4 |
| SLS | 23.8 |
| SLE3S | 33.0 |
| Kathon | 0.05 |
| Total | 99.85 |

An alternatively a shampoo formulation that includes 69% of a V-Chassis Surfactant Base, based on the total weight composition can be made. The V-Chassis formulation Surfactant Base includes the materials specified in Table 1b, below:

TABLE 1b

| V-formula Surfactant | |
|---|---|
| Base Ingredients | Wt. % |
| Water | 33.07 |
| SLS - neutralized and preserved | 15.79 |
| SLE3S - neutralized and preserved | 20.01 |
| Total | 68.87 |

The formulation will further include a co-surfactant, specifically cocoamidopropyl betaine (abbr. cap. betaine). The formulation will also include other ingredients as well; however, for purposes of carrying out the inventive methods, it was sufficient to recognize that the aforementioned ingredients would have the greatest effect on the overall viscosity of the finished formulation.

Among the foregoing materials, those most likely to contain components that vary from lot-to-lot that also effect viscosity were identified. It was determined that the molar sodium ion concentration in each of SLS (sodium lauryl sulfate) and SLE3S (sodium laureth sulfate, 3 moles of ethoxylation) varies and effects viscosity of the finished formulation. The amount of unreacted fatty alcohol present in each of these two surfactants also varies and affects the viscosity of the finished formulation. Further, the amount of sodium ion concentration in the co-surfactant also can be expected to vary from lot-to-lot. The variances are set out in Table 2, below:

TABLE 2

| Surfactant/ | | Low | High |
|---|---|---|---|
| Co-Surfactant | Component | (Wt. %) | (Wt. % |
| SLS | Fatty Alcohol | 0.37 | 0.6 |
| SLS | Na₂SO₄ | 0.08 | 0.5 |
| SLS | NaCl | 0.1 | 0.2 |
| SL3ES | Fatty Alcohol | 0.85 | 1.1 |
| SL3ES | Na₂SO₄ | 0.1 | 0.6 |
| SL3ES | NaCl | 0.005 | 0.4 |
| Cap. Betaine | NaCl | 5.43 | 6 |

### Determination of a Target Salt Concentration in the Final Composition

A number of preliminary calculations should be performed to determine the target salt concentration that will achieve a viscosity for the finished formulation that falls within an acceptable range (i.e., a target viscosity).

Four experiments were carried out to simulate the four permutations of having low and high salt content and low and high content of fatty alcohol:
Experiment 1: High Fatty Alcohol and High Salt
Experiment 2: Low Fatty Alcohol and Low Salt
Experiment 3: Low Fatty Alcohol and High Salt
Experiment 4: High Fatty Alcohol and Low Salt
   In each experiment, the surfactants employed each had contents of salt and fatty alcohol that coincided with the “Low” value reported in Table 2, above. For examples 1, 3, and 4, above, the amount of salt and/or fatty alcohol was modified by the addition of the pertinent salt or fatty alcohol based on Equation 1A, below:

$$\frac{X + \frac{low}{100} * batchsize}{batchsize + X} = \frac{high}{100} , \qquad Eqn . 1 A$$

where, “low” refers to the “Low” value in Table 1 for the associated component, “high” refers to the “High” value in Table 1 for the associated component, “batchsize” is the amount (in grams) of the particular surfactant (or cap. betaine), and “X” refers to the amount (in grams) of salt or fatty alcohol to supplement. Equation 1, rearranged to solve for “X” is shown as Equation 1B, below:

$$X = \frac{batchsize ( high - low )}{100 - high} \qquad Eqn . 1 B$$

The batch sizes of SLS and SL3ES were 191 grams and 263 grams, respectively. These batch sizes were determined by assuming an 800 gram batch and then multiplying that amount by the proportions in which each are present in the base, as reported in Table 1, above. Thus, an 800 gram batch of the base will contain about 191 grams of SLS (which is 800 multiplied by 23.8%), and 263 grams of SLE3S (which is 800 multiplied by 33%). An 800 gram batch of cap. betaine was used for the cap. betaine.

On the basis of the foregoing information, the Table 3, below, sets out the amount (X, in grams) of the particular salt and fatty alcohol that needs to be added to the Surfactant Base or the Vanilla Base depending upon which of Experiments 1 through 4 are being carried out. For Experiment 3, of course, no additional salt or fatty alcohol is necessary.

TABLE 3

| | Surfactant/ | Batchsize | X |
|---|---|---|---|
| Component | Co-Surfactant | (grams) | (grams) |
| Fatty Alcohol | SLS | 191 | 0.442 |
| Fatty Alcohol | SLE3S | 263 | 0.665 |
| Na₂SO₄ | SLS | 191 | 0.806 |
| Na₂SO₄ | SLE3S | 263 | 1.32 |
| NaCl | SLS | 191 | 0.19 |
| NaCl | SLE3S | 263 | 1.23 |
| NaCl | Cap. Betaine | 800 | 4.59 |

The compositions of the four experiments were prepared in a laboratory.

The salts were added to the compositions by first aggregating them and then dissolving them into a reserved amount of the water in the Surfactant Base or Vanilla Base. The maximum amount of sodium bisulfate and sodium chloride that could be dissolved was determined and then that amount was used in the Surfactant Base or Vanilla Base for the experiments that require high salt. The addition of salt to the composition is also referred to herein as spiking the composition with salt.

The fatty alcohol is challenging to add to the compositions, but was accomplished by first diluting the surfactants (SLS and SL3ES) and then raising the temperature to 49° C. prior to addition of the preservative, Kathon. The addition of fatty alcohol to the composition is also referred to herein as spiking the composition with fatty alcohol.

Thus four 800 gram compositions of the Surfactant Base/Vanilla Base were prepared based on the permutations of the high/low salt and fatty alcohol. The cap. betaine was either the 5.43 wt. % version of the 6 wt. % version.

Once the compositions were prepared, the viscosity of each was measured and associated to the known concentration of sodium ions and weight of fatty alcohol present in each composition. Because there are two sources of sodium ions in each composition, it is important to combine them; otherwise, the salts need to vary independently and the number of experimental compositions increases from four to eight. This assumes that both salts completely dissociate in the Surfactant Base or Vanilla Base. Equation 2, below, shows how to convert the two salts into moles of sodium ions. This can be accomplished by dividing the weight (in grams) by the molecular weight of each salt, keeping in mind that sodium bisulfate contributes two moles of sodium ions whereas sodium chloride contributes only one. The molecular weight of sodium chloride is 58.4 g/mol and the molecular weight of sodium bisulfate is 142.0 g/mol.

$$Moles of {Na}^{+} = \frac{NaCl , grams}{MW NaCl} + 2 * \frac{{Na}_{2} {SO}_{4} , grams}{MW {Na}_{2} {SO}_{4}} \qquad Eqn . 2$$

It is important to account for the salts already in the SLS and SLE3S as well. Moles of sodium ions can be calculated by first multiplying the weight % of salt contributed by each surfactant by the amount of surfactant employed in each experiment, and then using Equation 2, above, to calculate the total number of moles of sodium ions in the raw materials.

It is further necessary to calculate the number of moles of sodium ions introduced into the formulation with the cap. betaine. This calculation is similar to Equation 2, but differs in that there is no sodium bisulfate component in cap. betaine. In other words, divide the total number of grams of sodium chloride (from both the spike and the raw material) by the molecular weight of sodium chloride.

The total amount of fatty alcohol (in grams) consists of the amount that comes in with the SLS and SLE3S surfactants based on the plus the amount spiked in the experiment. This amount can be calculated by multiplying the weight % of fatty alcohol contributed by each surfactant by the amount of surfactant employed in each experiment and adding to that figure the amount of fatty alcohol spiked into the composition of each experiment.

Viscosity of each composition was measured on a TA Instrument AR2000 (manufactured by TA Instruments of New Castle, Del.). A steady state flow curve from 0.1 s⁻¹ to 100 s⁻¹ was measured, but only the value at 2 s⁻¹ was used. No additional salt was added because many of the experiments would have been above the upper limit without salt addition. This was learned in prior experiments by plotting the salt curve. The final range of viscosities was from 6 Pa·s (6,000 cPs) to about 21 Pa·s (21,000 cPs) at 25° C. and at a shear rate of 2 s⁻¹. The data obtained are reported in Table 4, below:

TABLE 4

| | Fatty Alcohol | Sodium Ions | Viscosity |
|---|---|---|---|
| Experiment | (Grams) | Molar Conc. | (cPs) |
| 1 | 3.2681 | 0.1192 | 21,370 |
| 2 | 2.3957 | 0.06981 | 6,027 |
| 3 | 3.2873 | 0.11748 | 14,430 |
| 4 | 2.3856 | 0.06969 | 10,020 |

These data were then employed in conventional statistical analysis software to determine an accurate equation for predicting the viscosity of a composition on the basis of the concentration of sodium ions and weight of fatty alcohol present in the composition. Specifically, the statistical analysis of the data was performed using the JMP 8® Fit Model platform, commercially available from JMP®, which is a wholly owned subsidiary of SAS® (a United States based statistical software company). The analysis can be performed readily by persons ordinarily skilled in the art of statistical data analysis. JMP® is a suitable software package because it is also accompanied by a prediction profiler capability, as explained below.

The inputs into the statistical analysis program are the molar concentration of sodium ions of the measured material, the weight of the fatty alcohol of the measured material, and the final viscosity.

A standard least squares approach was used with two (2) main effects and one (1) interaction term, also termed a “Factorial to Degree” fit model. The resulting prediction formula is set out below as Eqn. 3, which was saved as part of the original data table and listed in the JMP® data table:

Viscosity (cPs)=(257Y)+(31,897Z)+(60,921YZ)−7,004, Eqn. 3

wherein Y represents the grams of fatty alcohol and Z represents the moles of sodium ions.

The fit model can predict the viscosity based on the grams of fatty alcohol and molar concentration of sodium ions. The fit model has an R² value of 1, which means that the fit model will perfectly predict a shampoo of any given viscosity as long as it is made in interpolated space (i.e., within viscosity range of 5000 cPs to 21,000 cPs). The model becomes unreliable when extrapolation outside of that viscosity range is performed.

A JMP® prediction profiler was developed from this formula (fit model) using the “Graph, Profiler” functionality in JMP® (an algorithm proprietary to the software manufacturer). This prediction profiler was then used to explore the range of sodium ions and fatty alcohol content possible while still meeting the target viscosity value, and to determine optimal levels of fatty alcohol and total sodium ions required to achieve a target viscosity. This profiler allows one to predict where the limits are for total sodium ions and fatty alcohol content and still maintain a viable shampoo product based on established target viscosity and conductivity levels.

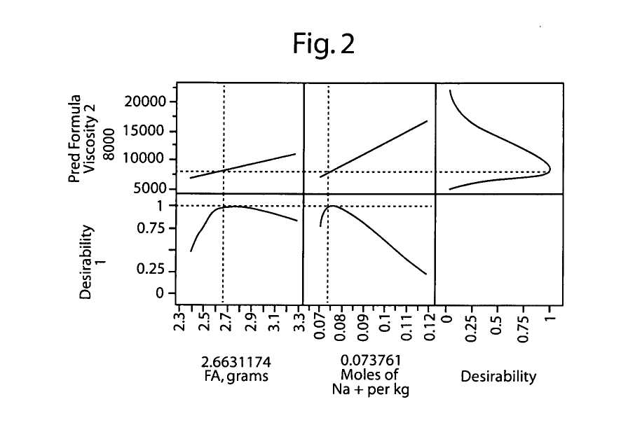

An example of the profiler is shown in FIG. 2. Shown there are five graphs on a single computer screen. A target viscosity desired for the finished shampoo formulation is set at 8000 cPs. A desirability value is set at 1 to maximize the chance that the target viscosity will be met. From those two data points, the profiler predicts the molar concentration of sodium ions and grams of fatty alcohol necessary in the final formulation to achieve the target viscosity. Deviations from the molar concentration of sodium ions and grams of fatty alcohol can be considered by the skilled artisan by reference to the profiler to determine how those deviations can be expected to affect the viscosity.

Often there are multiple responses measured and the desirability of the outcome involves several or all of these responses. For example, it may be desirable to maximize one response, minimize another, and keep a third response close to some target value. In desirability profiling, one specifies a desirability function for each response. The overall desirability can be defined as the geometric mean of the desirability for each response. Exemplified above is simply one of many ways to profile and predict viscosities relative to sodium ion concentration and fatty alcohol weight.

From the foregoing statistical analysis, a determination was made that the target sodium ion concentration desired in the finished shampoo formulation would be 0.074 moles Na⁺ per kilogram of formulation. That target value was determined to suitably ensure a viscosity within a range of 5,000 cPs to 12,000 cPs, desirable for many classes of conventional shampoo formulations.

Correlating Conductivity to Surfactant, Vanilla Base and Cap. Betaine

With a target salt concentration of 0.074 moles Na+ per kilogram of formulation on the basis of a minimum amount of fatty alcohol content, sample formulations were prepared on the basis of the composition of Experiment 2, described above. The sample formulations varied from one another in salt content. Specifically, salt was added to the compositions of each of Experiments 2 and 4 at 0%, 25%, 50%, 75% and 100% of the way to the maximum salt concentration in the SLS and SLE3S Surfactants and the Vanilla Base. Thereafter, the conductivity of each sample was measured. Equations 4a, b and c, below, were derived by linear regression of the data, and the equation was essentially the same for both of the compositions of Experiments 2 and 4:

Conductivity (mS/cm)=45.4X+38.3, Eqn. 4a SLS

Conductivity (mS/cm)=52.6X+31.7 Eqn. 4b. SLE3S

Conductivity (mS/cm)=60.6X+19.994 Eqn. 4c Vanilla Base

The number of moles of sodium ions per kilogram of Surfactant can be determined by solving for X in Equation 4a or 4b, above or similarly for the Vanilla Base using equation 4c. The results of these measurements showed two things. One, that the fatty alcohol content had no impact on the conductivity measurement as demonstrated by an essentially same equation for each of the compositions of Experiments 2 (low fatty alcohol content) and 4 (high fatty alcohol content). Thus, if you have two Surfactants or Vanilla Bases that are identical except for the fatty alcohol concentration the conductivities will be the same. Two, that the conductivity and salt concentration, measured in moles of sodium ions, is correlated with a good R² values of 0.9999.

Similar analyses were carried out for the cap. betaine. For those analyses, sodium chloride was dissolved in the cap. betaine in equal amounts between the 5.43% starting level up to the maximum amount at 6%, and thereafter the conductivity was measured. Thereafter, the conductivity was measured. Equation 5, below, was derived by linear regression of the data:

Conductivity (mS/cm)=6.3152X+11.764, Eqn. 5

The weight % sodium chloride present in the cap. betaine can be determined by solving for X in Equation 5, above. Thereafter, the weight % sodium chloride was converted into moles of sodium by first calculating the number of grams of sodium chloride and then dividing that amount by the molecular weight of sodium chloride (58.4 g/mol). The conductivity results for cap. betaine showed a correlation between salt content and conductivity having a good R² value of 0.98.

### Associating the Measurements to the Target Salt Concentration and Determining Additional of a Suitable Viscosity Modifier

As stated above, the target salt concentration for the finished shampoo formulation was determined to be 0.074 moles Na⁺ per kilogram of formulation. The molar concentration of sodium ions calculated from the conductivity measurements made on the Surfactant or the Vanilla Base and cap. betaine, described immediately above, were aggregated and compared to the target salt concentration. The difference between the aggregated value and the target value informs the manufacturer of how much viscosity modifier to supply. The difference is multiplied by the molecular weight of sodium chloride (58.4 g/mol) to provide a value of sodium chloride in grams per kilogram of formulation that should be added to achieve the target.

Where the target value exceeds the aggregated value, sodium chloride in this amount should be added. Where the aggregated value exceeds the target value, a hydrotrope should be added.

Data in Table 5, below, were obtained from four validation experiments that demonstrate that the conductivity may be reliably employed to determine the sodium ion concentration in the final product and to achieve an acceptable target viscosity:

TABLE 5

| | | | Surfactant or Vanilla | | | | | | | | | | | | |
|---|---|---|---|---|---|---|---|---|---|---|---|---|---|---|---|
| | | | Base | | | | Cap. Betaine | | | | | | | | |
| Target | | | Con- | mol | | | Con- | | | | Total in | | | | Final |
| mol | Batch | | duc- | Na⁺/kg | kg of | | duc- | | kg of | | Sur- | Run Specific Material | | | Viscosity, |
| Na⁺/kg | size, | mol | tivity, | Vanilla | Vanilla | mol | tivity, | wt. % | Cap. | mol | factants | mol | grams | grams | cP |
| shampoo | kg | Na⁺ | mS/cm | Base | Base | Na⁺ | mS/cm | NaCl | Betaine | Na⁺ | mol Na+ | Na⁺ | NaCl | SXS | @2/s |
| 0.074 | 1 | 0.074 | 20.833 | 0.0122 | 0.65 | 0.0076 | 45.468 | 5.34 | 0.06667 | 0.061 | 0.069 | 0.0045 | 0.263 | 0 | 10,500 |
| 0.084 | 1 | 0.084 | 20.641 | 0.0107 | 0.65 | 0.0070 | 45.468 | 5.34 | 0.06667 | 0.061 | 0.068 | 0.016 | 0.942 | 0 | 9,200 |
| 0.084 | 1 | 0.084 | 20.635 | 0.0106 | 0.65 | 0.0069 | 45.468 | 5.34 | 0.06667 | 0.061 | 0.068 | 0.016 | 0.942 | 0 | 12,800 |
| 0.084 | 1 | 0.084 | 23.912 | 0.0646 | 0.65 | 0.0422 | 49.258 | 5.94 | 0.06667 | 0.068 | 0.11 | 0 | 0 | 5.32 | 7,400 |
| 0.084 | 1 | 0.084 | 49.258 | 0.0541 | 0.65 | 0.0353 | 49.258 | 5.94 | 0.06667 | 0.068 | 0.10 | 0 | 0 | 3.83 | 10,400 |

For exemplary purposes, the values reported in sample formulation were determined according to the following calculations consistent with the foregoing description:

1. Measured Conductivity: 20.641 mS/cm. Using Equation 4, this is 0.0107 moles of Na⁺/kg of Vanilla Base. Multiply by the amount of Vanilla Base in this formula, 0.65 kg, to get 0.0070 moles of Na⁺.

2. Measured conductivity: 45.468 mS/cm. Using Equation 5, this is 5.34 wt. % NaCl. Take 5.34% of the cap. betaine, 0.06667 kg and then divide by the molecular weight to get 0.025 moles of Na⁺.

3. Now, take 0.084-0.0070-0.025 to get 0.016 moles of Na⁺ remaining

4. Convert this to grams of NaCl required by multiplying by the molecular weight of NaCl (58.4 g/mol). This means 0.942 grams of NaCl need to be added to the 1 kg batch. The final viscosity was measured to be 9,200 cPs at 2/s—within the target range of 5,000 cPs to 12,000 cPs.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method comprising:
(a) combining ionic surfactant-containing feeds in a mixer to form a composition selected from the group consisting of a personal care composition, a laundry detergent, and a cleaning composition;
(b) determining the concentration of ions necessary in the composition to achieve a composition viscosity;
(c) measuring the conductivity of one or more ionic surfactant-containing feeds upstream of the mixer;
(d) correlating the measured conductivity to the concentration of ions present in the measured ionic surfactant-containing feeds; and,
(e) introducing a viscosity modifier to the mixer in a quantity per unit flow of the composition sufficient to achieve the composition viscosity.

2. The method of claim 1, wherein the composition is a personal care composition selected from the group consisting of a shampoo composition, shower gel, liquid hand cleanser, liquid dental composition, skin lotion and cream, hair colorant, facial cleanser, and fluids intended for impregnation into or on wiping articles.

3. The method of claim 2, wherein the composition is a shampoo composition.

4. The method of claim 1, wherein the ions comprise cations.

5. The method of claim 1, wherein the mixer is selected from the group consisting of an agitated mixer, an orifice, a homogenizer, an in-line dynamic mixer, a high pressure sonic mixer, and a static mixer.

6. The method of claim 1, wherein combining comprises continuously flowing the feeds to the mixer.

7. The method of claim 6, wherein combining further comprises simultaneously introducing the flowing feeds to the mixer.

8. The method of claim 1, wherein measuring the conductivity comprises measuring at a temperature of about 20° C. to about 45° C.

9. The method of claim 1, wherein correlating comprises determining the number of cations present in the ionic surfactant-containing feeds based on the measured conductivity and correlating that number to the concentration of cations necessary in the composition to achieve the composition viscosity.

10. The method of claim 1, wherein each of the ionic surfactant-containing feeds individually comprises one or more surfactants selected from the group consisting of an amphoteric surfactant, an anionic surfactant, and a Zwitteronic surfactant.

11. The method of claim 1, wherein the viscosity modifier comprises one or more compounds that liberate a cation in the presence of the ionic surfactant-containing feed, the cation selected from the group consisting of calcium, potassium, sodium, lithium, ammonium, and tetraethylammonium (TEA).

12. The method of claim 1, wherein the viscosity modifier is a hydrotrope selected from the group consisting of C₁₋₈alkyl carboxylates, C₁₋₈alkyl sulfates, C₁₋₈alkyl benzene sulfonates, halogen benzoates (e.g., chlorbenzoate), C₁₋₈alkyl naphthalene carboxylates, (e.g., hydroxyl naphthalene carboxylate), urea, ethoxylated sulfates, and mixtures thereof.

13. The method of claim 1, wherein the viscosity modifier is a polymeric thickener selected from the group consisting of carboxylic acid polymers, crosslinked polyacrylate polymers, polyacrylamide polymers, and mixtures thereof.

14. The method of claim 1, wherein the personal care composition further comprises one or more ingredients selected from the group consisting of water, oily conditioning agents, natural cationic deposition polymers, synthetic cationic deposition polymers, anti-dandruff agents, particles, suspending agents, paraffinic hydrocarbons, propellants, dyes, non-volatile solvents or diluents, pearlescent aids, foam boosters, nonionic cosurfactants, pediculocides, pH-adjusting agents, perfumes, preservatives, chelants, proteins, skin active agents, sunscreens, UV absorbers, and vitamins.

15. The method of claim 1, wherein the composition viscosity is about 2.5 Pascal seconds (Pa·s) to about 100 Pa·s determined at 25° C. and at a shear rate of 2 per second (s⁻¹).

16. A continuous method of making a personal care composition, the method comprising
(a) simultaneously combining continuous flows of ionic surfactant-containing feeds and viscosity modifier selected from the group consisting of an ion source, a hydrotrope, and a polymeric thickener to form a personal care composition having a viscosity in a range of about 2.5 Pa·s to about 100 Pa·s at 25° C. and at a shear rate of 2 s⁻¹;
(b) measuring the conductivity of one or more ionic surfactant-containing feeds prior to formation of the composition, and,
(c) correlating the measured conductivity to an intermediate salt concentration,
wherein the flow of the viscosity modifier is at a rate per unit flow of the ionic surfactant-containing feeds sufficient to achieve a salt concentration in the formed personal care composition corresponding to the viscosity range.

17. In a continuous manufacture of a composition selected from the group consisting of a personal care composition, a laundry detergent composition, and a cleaning composition, a method of adjusting the viscosity of the composition, the method comprising:
(a) measuring the conductivity of one or more ingredients employed in the manufacture;
(b) correlating the measured conductivity to an intermediate salt concentration of the composition;
(c) mixing with the ingredients a quantity of a viscosity modifier sufficient to change the intermediate salt concentration of the composition to a target salt concentration sufficient to achieve a target composition viscosity.

18. The method of claim 17, wherein the ingredients comprise surfactants.

19. The method of claim 17, wherein correlating further comprises determining the difference between the target salt concentration and the intermediate salt concentration.

20. The method of claim 19, wherein the quantity of viscosity modifier mixed with the ingredients is a function of the difference between the target salt concentration and the intermediate salt concentration.
